(19) United States
(12) Patent Application Publication
(10) Pub. No.: US 2018/0070982 A1
(43) Pub. Date: Mar. 15, 2018

(54) Magnetic Resonance Compatible RF Transseptal System

(71) Applicant: Imricor Medical Systems, Inc., Burnsville, MN (US)

(72) Inventors: Scott Kimmel, Roseville, MN (US); Thomas W. Lloyd, Eagan, MN (US); Milton N. Turcios, Farmington, MN (US)

(21) Appl. No.: 15/575,647

(22) PCT Filed: Jun. 15, 2016
(86) PCT No.: PCT/US16/37572

§ 371 Date: Nov. 20, 2017

Related U.S. Application Data
(60) Provisional application No. 62/181,569, filed on Jun. 18, 2015.

(51) Int. Cl. A61B 17/34; G01R 33/28

(52) U.S. Cl. CPC A61B 17/34; A61B 2017/00247; G01R 33/285

(57) ABSTRACT

An MR compatible RF transseptal needle system is provided. The transseptal needle system includes a transseptal needle having a main cannula portion and a distal tip portion; and a delivery tool with a distal tip electrode. In some embodiments the transseptal needle system further includes a linear position measurement mechanism including a wiper member; and two or more conductive strip members.

### FIELD OF INVENTION

This invention relates to actively-tracked medical devices. More particularly, the present invention is related to a transseptal system used in interventional vascular procedures and which may be actively visualized and/or tracked in a magnetic resonance imaging (MRI) environment.

### BACKGROUND OF THE INVENTION

MRI has achieved prominence as a diagnostic imaging modality, and increasingly as an interventional imaging modality. The primary benefits of MRI over other imaging modalities, such as X-ray, include superior soft tissue imaging and avoiding patient exposure to ionizing radiation produced by X-rays. MRI's superior soft tissue imaging capabilities have offered great clinical benefit with respect to diagnostic imaging. Similarly, interventional procedures, which have traditionally used X-ray imaging for guidance, stand to benefit greatly from MRI's soft tissue imaging capabilities. In addition, the significant patient exposure to ionizing radiation associated with traditional X-ray guided interventional procedures is eliminated with MRI guidance. Presently, however, due to the lack of appropriate surgical instrumentation MRI is not available to interventionalists for use during interventional therapy to accurately track and precisely guide medical devices to regions of a patient needing treatment.

By way of example, the left atrium of the heart is the most difficult cardiac chamber to access percutaneously. Although the left atrium may be reached via the left ventricle and mitral valve, manipulation of catheters requiring two 180 degree turns may be cumbersome and time consuming for the surgeon. Thus the transseptal puncture is the procedure of choice because it permits a direct route to the left atrium via the intra-atrial septum and systemic venous system. The technique has been used for mitral valvuloplasty and ablation in the left heart and with the explosion of interest in catheter ablation of atrial fibrillation, transseptal puncture is increasingly being adopted by cardiac electrophysiologists and the method of choice. Another example of using a transseptal needle is puncturing of a ventricular wall preceding, for example, chemo-ablation for ventricular tachycardia. It is critical for cardiac electrophysiologists and other electrophysiologists interested in transseptal puncture techniques to be able to track sheaths for carrying dilators and transseptal puncture devices in an MR environment in order to know when the transseptal device has punctured the septum and to avoid unintentional perforation of adjacent structures such as vessel walls and the like.

While there are many types of sheaths, dilators and transseptal needles currently available for transseptal puncture (and other medical procedures) few are well-suited for use in an MRI environment and to the inventor's knowledge none are actively tracked. For example, deflectable (i.e., steerable) sheaths including multi-directional, bi-directional and uni-directional deflectable catheters are known. However, many of these sheaths have ferromagnetic components that pose a safety hazard to the patient in a magnetic field environment, as they can cause injury to the patient due to undesired movement caused by the magnetic field. The ferromagnetic components can also cause image distortions, thereby compromising the effectiveness of the procedure. Still further, such sheaths may include metallic components that may cause radiofrequency (RF) deposition in adjacent tissue and, in turn, tissue damage due to an extensive increase in temperature.

Similarly, transseptal needles currently available have the same problems. Notably, they either include ferromagnetic components that cause image distortions or include metallic components that cause RF deposition in tissue.

Moreover, it is difficult to track and/or visualize the location of the aforementioned sheaths, dilators and transseptal needles in an MRI environment. In general, there are two types of tracking in an MRI environment: active tracking and passive tracking. Active tracking is more robust than passive tracking but typically involves resonant RF coils that are attached to the device and directly connected to an MR receiver allowing for the determination of the three-dimensional coordinates of the resonant RF coils within the scanner. To the inventors' knowledge neither active nor passive tracking techniques are presently utilized in conventional sheaths, dilators or transseptal needles.

Conventional transseptal needles are used to the puncture the septum in a mechanical manner. That is, these needles have a sharpened metal tip that will pierce the septum when an advancing force is applied to the transseptal needle handle. Although the mechanical technique has a long track record of success, for a small percentage of patients, those with an elastic, aneurysmal, or thickened septum due to fibrosis, it is less effective. Additionally, there are safety concerns associated with the mechanical needle due to the potential of the sharpened tip to skive the inner surface of the dilator and create plastic particles, which could lead to thromboembolism. For the aforementioned reasons, an RF needle has been developed. The RF needle utilizes the application of energy to burn a hole through the septum and has the potential to allow for more effective and quicker punctures of elastic, aneurysmal, and thickened, fibrotic septa. In addition, a RF needle can be designed with a less sharp or blunt tip, which could eliminate dilator skiving and the associated generation of thromboembolitic plastic particles. Although the RF needle has the aforementioned advantages, it is unsuitable for the MR environment due to the presence of ferromagnetic components, which, as described above, create safety hazards.

Thus, there is a need for an RF transseptal system that can be actively and effectively tracked and/or visualized in an MRI environment.

### BRIEF SUMMARY OF THE INVENTION

The invention described here is an MR compatible RF (MR-RF) transseptal system that includes a transseptal needle, a dilator and a deflectable sheath. Numerous structural variations of an MR compatible steerable sheath and dilator that can be actively tracked in accordance with the invention are contemplated and within the intended scope of the invention. Those of skill in the art will appreciate that the exemplary actively tracked sheath and/or dilator can be accomplished in a variety of ways. Those of skill in the art will also appreciate that the transseptal puncture device may comprise numerous structural variations. Therefore, for purposes of discussion and not limitation, exemplary embodiments of the MR compatible RF transseptal system will be described in detail below.

In a first aspect of the MR-RF system, there is a transseptal needle that utilizes an MRI safe material, such as a polymer tube, for the majority of its length, or main cannula portion. At the distal tip end of the main cannula, there is a conductive material forming the tip of the needle that extends from and is bonded to the main cannula. The conductive metal tip may be bonded to the main cannula portion with a method such as adhesive bonding, overmolding, crimping, or some other mechanical means. The conductive material may be a metal such as stainless steel or a non-ferromagnetic conductor, such as aluminum, platinum, nitinol, inconel, or the like.

In order for RF power to be delivered to the conductive tip of the needle, the needle works in conjunction with a delivery tool such as a dilator or sheath. The delivery tool includes an electrical contact located in the distal portion of the inner lumen, which receives the needle. The electrical contact may comprise a ring electrode, conductive pad, or the like. An electrical connection between the needle and delivery tool is made when the needle is advanced to the point at which the conductive portion of the needle tip makes contact with the electrical contact within the inner lumen of the delivery tool. This may be facilitated by creating an interference fit between the electrical contact and the outer surface of the conductive portion of the needle. This connection point could be a discrete point, such as when the needle is fully extended from the delivery tool, or it could be multiple continuous points, such as from the moment the needle tip just exits the delivery tool to the moment of maximum needle extension.

To provide MR safety, the delivery tool includes an MRI safe means of conducting RF energy to the electrical contact in the delivery tool and subsequently to the needle. Examples of an MRI safe means of delivering RF power are described in U.S. Pat. Nos. 8,588,934 and 8,588,938.

When the conductive portion of the needle is in contact with the electrical contact of the delivery tool, RF energy can be transmitted through the MRI safe wire assembly to the tip of the needle. This RF energy can then either be used to make the septal puncture alone as is the case with the existing RF transseptal needle, or work in conjunction with the mechanical puncture force of the needle to make the septal puncture.

The transseptal needle may be either hollow or solid. A hollow needle may be used to provide a needle construction similar to traditional needles thus allowing the needle to be used with only mechanical force to puncture the septum and without the specialized delivery tool disclosed herein.

A solid needle may be used to increase the mechanical strength, improve manufacturability, or reduce the diameter of the needle.

The transseptal needle tip may be sharpened or rounded. The benefit of the sharpened tip is that the needle can be used solely for a mechanical puncture if the RF is not needed or desired. The disadvantage, as mentioned previously, is that the sharpened tip has the potential to skive the delivery tool inner surface and create thromboembolitic plastic particulate matter. The benefit of the rounded tip is that it will not skive the inner surface of the delivery tool. The disadvantage of the rounded tip is that the needle cannot be used solely for a mechanical puncture.

The RF transseptal system may include a temperature sensor to monitor the temperature of the needle tip during RF energy application. One example of an MR safe means of monitoring temperature is the use of a fiber optic temperature sensor. The sensor may be placed in the lumen of the needle. Alternatively, the temperature sensor may reside in the delivery tool and include a thermal contact to the needle.

Regardless of placement, the temperature sensor may be removable allowing for pressure monitoring, contrast injection, or another use of the lumen when temperature monitoring is not desired. Alternatively, the temperature sensor may be permanently attached to the needle.

Application of RF energy to the needle may be automatically triggered when the needle is fully deployed or manually controlled by the clinician.

In another aspect of the MR-RF needle, a linear position measurement mechanism (LPMM) may be used as a means for determining the location of the needle tip within the distal portion of the delivery tool. The LPMM consists of a wiper element on the needle that electrically couples two or more resistive conductive strips or elements on the delivery device. The LPMM could be located proximally or distally on the needle and delivery tool. If the LPMM were located proximally, the respective elements would reside in the handle or hub elements of the needle and delivery tool. If the LPMM were located distally, the respective elements would reside on the distal outer surface of the needle and the distal inner surface of the delivery tool.

The wiper element functions to electrically connect the resistive, conductive elements creating an electrical loop. At the beginning and end of the loop are measuring points. The resistance measured at these measuring points increases or decreases as the wiper slides along the elements.

The resistive elements may have a linear or logarithmic electrical characteristic. An exemplary embodiment of a linear resistive element is one with a constant cross-section, causing the resistance between the wiper contact and the element to be proportional to the distance.

An exemplary embodiment of a logarithmic resistive element is one with cross-section that tapers. In another embodiment, the resistance of the element changes from one end to the other due to the fact that the element is constructed of multiple materials with differing resistivity. In both embodiments, the resistive output is a logarithmic function to the wiper position.

The LPMM could be incorporated into the MR-RF transseptal needle system by placing the two resistive linear elements on the delivery tool handle. A conductive wire would be electrically connected to the proximal end of each element. The wiper element would then be placed on the handle of the needle. When the needle is inserted far enough into the delivery tool, the wiper element makes electrical contact with the resistive elements. This contact creates an electrical short between the two resistive elements and the impedance of the circuit formed by the elements and the wiper can be measured. As the needle is advanced, a greater portion (length) of the resistive elements is contained within the circuit formed by the elements and the wiper and the impedance of the circuit changes. When the needle retracts, a shorter length of the resistive elements is contained with the circuit and the change in impedance is reversed.

For the purpose of calibrating the needle location or determining the impedance presented when the needle is fully retracted, a mechanism for creating an electrical short between the proximal ends of the elements may be used.

Those of skill in the art will appreciate that the elements of the LPMM could be switched so that the wiper element is incorporated into the delivery tool and the resistive strips are incorporated in to the needle.

In another embodiment of the MR-RF transseptal needle, an MR compatible injection catheter may be used as the delivery device for the MR Compatible RF Transseptal Needle, thus forming an MR Compatible RF Injection Catheter.

The MR Compatible RF Injection Catheter would work in a similar fashion where RF energy may be applied to the injection needle to reduce the force required to advance the needle into tissue. This could be especially advantageous during puncturing of a ventricular wall proceeding, for example, chemo-ablation for ventricular tachycardia. It would also allow the needle to have a thinner wall because less puncture force requires less cannula column strength. A thinner wall could reduce the overall outer diameter of the injection catheter. Otherwise, a thinner wall could mean a larger inner diameter for the cannula. A larger cannula inner diameter would be beneficial for an injection catheter that would deliver a viscous solution such as that used for stem cell delivery. This is because an increased inner diameter creates less friction for the injected fluid.

The needle of the RF injection catheter may be advanced through a distal electrode of the injection catheter. In this case, the needle may be electrically isolated from the electrode as well as any other electrodes to reduce or eliminate delivery of RF energy through the electrode(s) and electrical communication between the needle and the electrodes.

An MR compatible deflectable sheath is another example of a potential delivery device for the MR compatible RF transseptal needle in accordance with the invention.

When using a deflectable sheath, it is advantageous to be able to characterize the deflection of the sheath. This is true for any deflectable sheath and not specifically limited to a sheath used in conjunction with a transseptal needle.

In another embodiment of the MR-RF system, the deflection of a sheath may be measured and characterized by incorporating a fiber optic Bragg sensor into the distal portion of the sheath. One method of accomplishing this is for the Bragg sensor to be disposed within the wall of the sheath. When the sheath is deflected, the Bragg sensor also deflects or bends and thereby the amount of sheath deflection is captured.

Measurement and characterization of the deflection is important because often times the amount of deflection distally, especially with an in-dwelling catheter, does not correlate with the clinicians expectation based on the amount of proximal handle actuation. For example, when the clinician has rotated the sheath knob fully, he/she expects that there is full deflection distally, but in many cases it is not fully deflected. Visualizing the amount of actual distal sheath deflection on a display may help the clinician better steer and maneuver the sheath. The distal curve deflection may be displayed to the clinician as a curve graphic or an alphanumeric symbol such as the angle of deflection.

Characterization of the deflection may be continuous and include the entire range of deflection or discrete and limited to specific points in the range of deflection.

Characterization of the deflection may also be limited to a single point in the range of deflection, such as the neutral position, or the configuration corresponding to when the distal section of the sheath is straight. This is useful during sheath removal from the patient.

Information regarding the deflection of the sheath could be relayed to the clinician by an indicator in the sheath handle, such as a LED. The color displayed by this LED could convey important information For example, the indicator could turn green to communicate that the sheath is straight and is safe for removal. The indicator could turn red to communicate that the sheath is deflected and is not safe for removal.

In another embodiment of the MR-RF transseptal system, the needle puncture force is measured by incorporation of a fiber optic Bragg sensor into the needle. Those of skill in the arts will appreciate that the Bragg sensor could also be incorporated into the delivery device, either the sheath or the dilator.

In the case of the transseptal needle, the tip of the needle could be slidably connected to the main cannula. The proximal surface or edge of the tip would be connected to the Bragg sensor, which would run the length of the inner lumen of the main cannula. When the puncture step is performed, the tip of the needle would translate proximally slightly within the lumen of the main cannula, causing a bend in the distal section of the fiber optic Bragg sensor. The amount of bending of the Bragg sensor would then correlate to magnitude of the puncture force. The fit between the needle tip and main cannula could be designed to be a friction fit so that the column strength of the fiber optic Bragg sensor is not being relied upon to hold the needle tip in place. Additionally, an ancillary tubing member could run along the length of the main cannula next to the fiber optic Bragg sensor and provide additional holding force for the needle tip. Those of skill in the arts will appreciate that the Bragg sensor could also be located at the proximal end of the cannula.

Similar to the transseptal needle, the Bragg sensor could be employed in an ablation catheter for contact force measurement. In this case, the needle tip is replaced with the distal tip electrode and the electrode is slidably connected to the catheter tip support. The fiber optic Bragg sensor is connected to the proximal edge of the distal tip electrode. When the distal tip electrode contacts tissue, it translates proximally within the tip support and causes the Bragg sensor to bend. The amount of bend is correlated to tip contact force.

### BRIEF DESCRIPTION OF THE DRAWINGS

For a better understanding of the invention, and to show how the same may be carried into effect, reference will now be made, by way of example, to the accompanying drawings, in which:

FIG. 1 is a perspective view of the transseptal needle component of the transseptal system in accordance with the invention wherein a metal tip is shown.

FIG. 2 is a view showing a dilator and an inserted transseptal needle in which the transseptal needle is not fully advanced and the metal portion of the needle is not making contact with a ring electrode in the dilator.

FIG. 3 is a view showing a dilator and an inserted transseptal needle in which the transseptal needle is fully advanced and the metal portion of the needle is making contact with the ring electrode in the dilator.

FIG. 4 is a view of a deflectable sheath component of the transseptal system in accordance with the invention showing a fiber optic Bragg sensor disposed in the wall of the deflectable sheath.

FIG. 5 is a view of the deflectable sheath of FIG. 4 showing that when the deflectable sheath bends the fiber optic Bragg sensor also bends.

FIG. 6 is a view of a transseptal needle with a fiber optic bragg sensor incorporated.

FIG. 7 is a view of the puncture step in which tip of the transseptal needle of FIG. 6 translates proximally thereby causing the fiber optic Bragg sensor to bend.

FIG. 8A is a top view illustration of a linear position measurement mechanism in which a wiper is used to electrically connect two resistance elements.

FIG. 8B is a front view of the linear position measurement mechanism shown in FIG. 8A.

FIGS. 9 and 10 are top view illustrations of the linear position measurement mechanism design in which the resistance measured at the measuring points increases or decreases as the wiper moves along the resistance elements.

FIG. 11 is a graph depicting the resistance as a function of the distance of wiper element along the resistance elements.

FIG. 12A is a proximal side view and FIG. 12B is a proximal top view of the incorporation of one half of the linear position measurement mechanism, the two resistive elements from FIG. 8A, into a delivery tool.

FIG. 13A is a proximal side view and FIG. 13B is a proximal top view of the incorporation of one half of the linear position measurement mechanism, the wiper element from FIG. 8A, into a transseptal needle.

FIG. 14A is a top view and FIG. 14B is a side view showing the needle in a first position in which a first length of the resistive elements is contained within the circuit formed by the linear position measurement mechanism.

FIG. 15A is a top view and FIG. 15B is a side view showing the needle in a second position more advanced than FIG. 14A and FIG. 14B in which a second, larger length of the resistive elements is contained within the circuit formed by the linear position measurement mechanism.

### DETAILED DESCRIPTION OF THE DRAWINGS

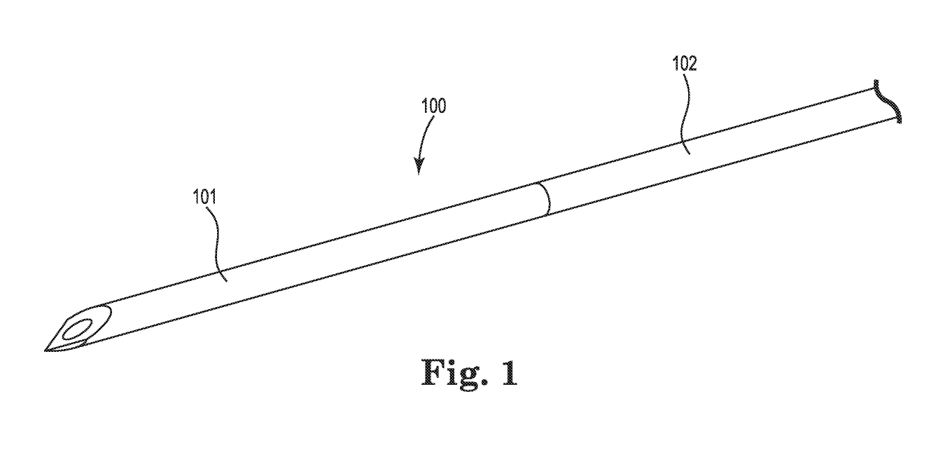

Referring now to the figures, FIG. 1 shows the distal end 100 of the MR-RF transseptal needle cannula. The cannula includes a tip portion 101 and a main cannula portion 102. The tip portion is made of a conductive material. The conductive material may be a metal such as stainless steel or a non-ferromagnetic conductor, such as aluminum, platinum, nitinol, inconel, or the like. The main cannula portion is made of an MR safe material, such as a polymer tube. The tip may be bonded to the main cannula portion via over molding, adhesive bonding, crimping, or some similar method.

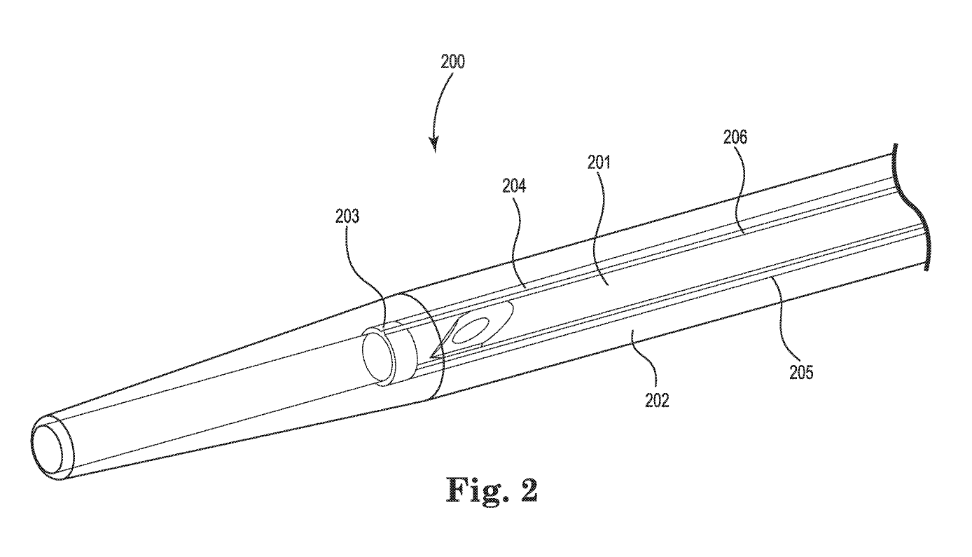

FIG. 2 shows the distal section 200 of the MR-RF design that includes the transseptal needle and the delivery tool. The transseptal needle tip 201 is in the retracted position within the inner lumen 204 of the delivery tool 202. Also within the inner lumen 204 is the ring electrode 203 and a first 205 and second 206 conduction line. Those of skill in the arts will also appreciate that the conduction lines 205 206 could be disposed within the wall of the delivery tool 202.

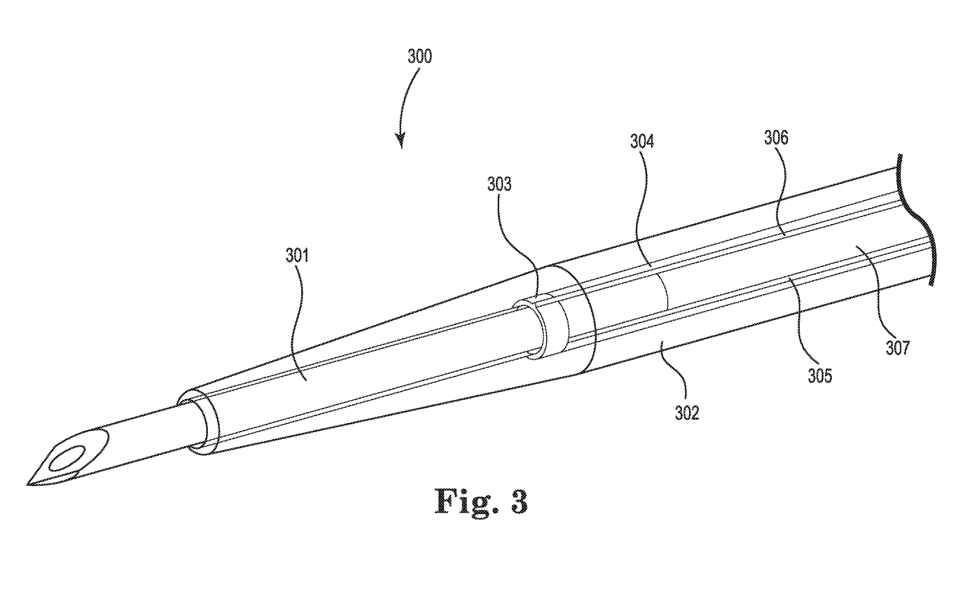

FIG. 3 shows the distal section 300 of the MR-RF design that includes the transseptal needle and the delivery tool. The transseptal needle tip 301 is in the extended position within the inner lumen 304 of the delivery tool 302. The main cannula section of the transseptal needle 307 is also now visible. In the extended position, the needle tip 301 comes into contact with the ring electrode 303. The first 305 and second 306 conduction lines are connected to the ring electrode 303. When energy is applied to the conduction lines 305 306, it is transferred into the ring electrode 303 and then further transferred to the needle tip 301. If the needle tip 301 is in contact with the septum, the energy will be burn the tissue, facilitating septal puncture.

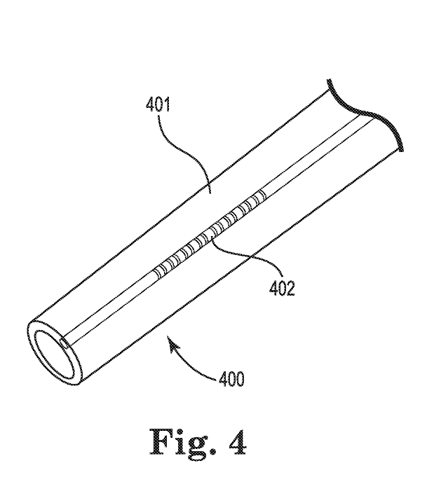

FIG. 4 shows the distal section 400 of the deflectable sheath 401 with a fiber optic bragg sensor 402 disposed within the wall of the sheath. In this view, the sheath is straight and therefore the bragg sensor is also straight.

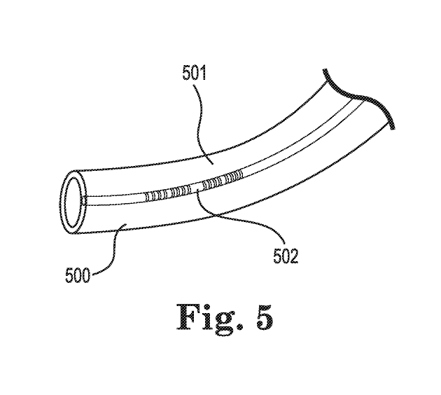

FIG. 5 shows the distal section 500 of the deflectable sheath 501 with a fiber optic bragg sensor 502 disposed within the wall of the sheath. In this view, the sheath is deflected and therefore the bragg sensor is also deflected. The amount of bragg sensor deflection can be quantified and correlates with the amount of sheath deflection. In this manner, the amount of sheath deflection is communicated to the clinician.

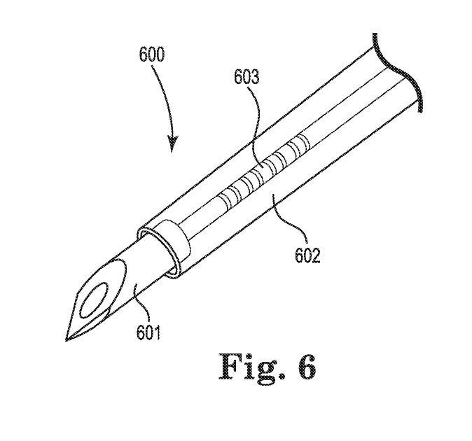

FIG. 6 shows the distal section of the transseptal needle 600. The tip of the transseptal needle 601 is slidably connected to the main cannula 602. The proximal edge of the transseptal needle 601 is connected to the distal edge of a fiber optic bragg sensor 603. The needle tip is pictured with a sharpened edge and as hollow, but those of skill in the arts will appreciate that tip shape could take many forms including less sharp or completely rounded. Also, the tip could be of a solid construction.

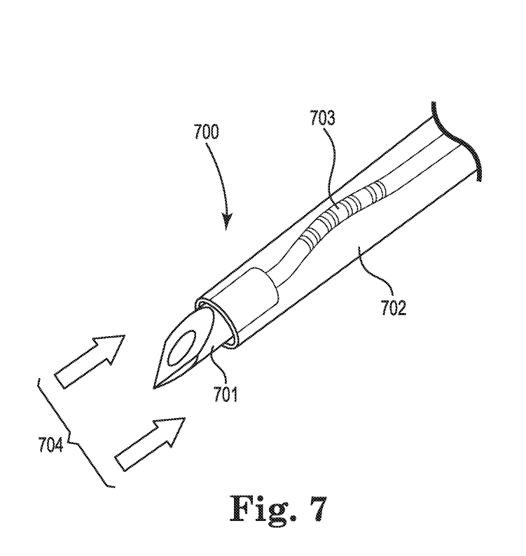

FIG. 7 shows the distal section of the transseptal needle 700 during the puncture step. When the puncture step is performed, the septal resistance force represented by the two arrows 704 pushes back slightly on the transseptal needle tip 701. Since the needle tip 701 is slidably connected to the main cannula 702, the septal resistance force 704 causes the needle tip 701 to translate slightly in the proximal direction, which is indicated by the arrows 704. The proximal surface of the needle tip 701 is connected to the distal edge of the fiber optic bragg sensor 703 and therefore the proximal translation of the needle tip 701 causes the bragg sensor 703 to bend. The amount of sensor bend can then be quantified and correlated to septal puncture force.

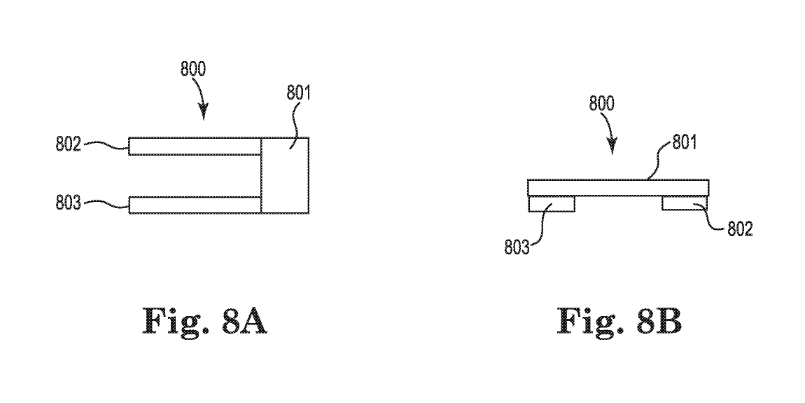

FIG. 8A is a top view of a linear position measurement mechanism (LPMM) in which a wiper element 801 connects two resistive elements 802, 803. The wiper element 801 can slide back and forth in the horizontal direction on the two resistive elements 802, 803. During this sliding movement, the wiper element maintains electrical contact with the resistive elements.

FIG. 8B is a front view of a LPMM in which a wiper element 801 connects two resistive elements 802, 803. This view shows that the wiper element 801 is located above the two resistive elements 802, 803. Those of skill in the arts will appreciate that the wiper element could also be located below, in between, or in some other connecting position between the two resistive elements.

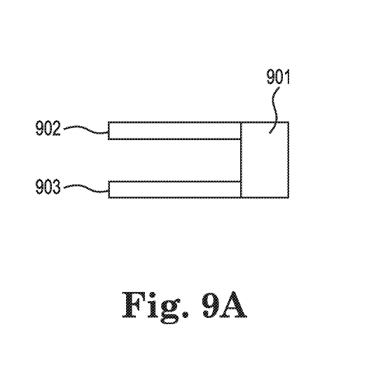

FIG. 9 is a top view of the LPMM in which the wiper element 901 is in a first position in relation to the resistive elements 902, 903. If an electrical loop were created by connecting a load to the left edges of the resistive elements, a long length of the resistive elements would be contained within the electrical loop. This would translate to a certain measured resistance value.

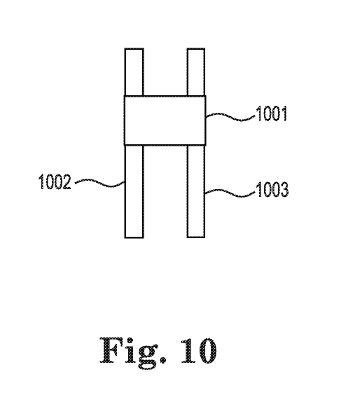

FIG. 10 is a top view of the LPMM in which the wiper element 1001 is in a second position in relation to the resistive elements 1002, 1003. As is the case in FIG. 9, an electrical loop is created, but since the wiper has moved to the left, less of the resistive elements would be contained within the electrical loop. This would translate to a different measured resistance value. Thus when an electrical loop is created, the measured resistance is a function of the wiper linear position and this is how the LPMM captures linear position.

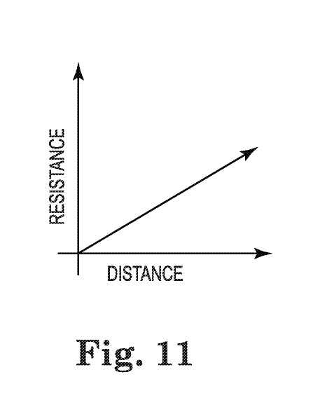

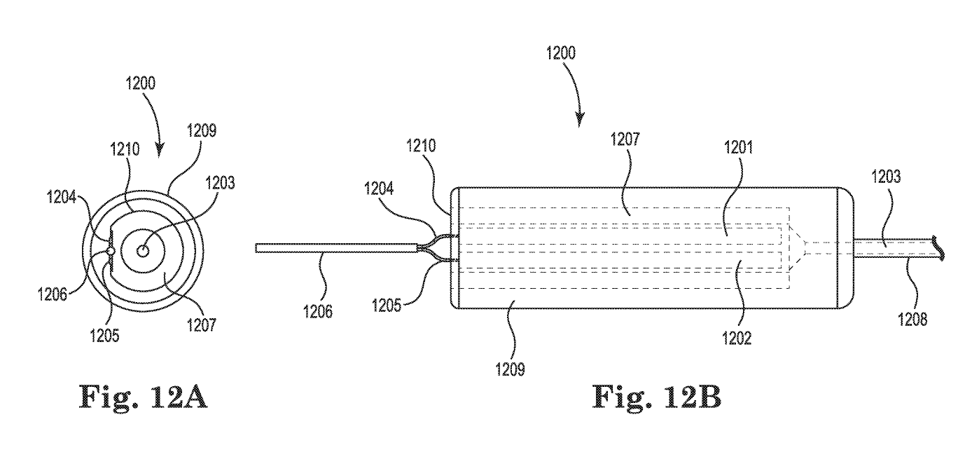

FIG. 11 is an example graph illustrating the relationship between the distance or linear position of the wiper element and resistance. In this case, the relationship is linear, but the relationship could also be curvilinear, logarithmic, or some other shape depending on how the resistive elements are constructed.

FIG. 12A and FIG. 12B are the proximal top and proximal side view respectively of the incorporation of the LPMM's two resistive elements 1201 and 1202 into a delivery tool 1200. The resistive elements 1201 and 1202 are disposed on the inner surface of a receiving cavity 1207, which is located within the delivery tool handle 1209. The receiving cavity is continuous with the delivery tool lumen 1203 on one side and has an opening 1210 on the other side. The delivery tool lumen 1203 is the inner channel of the delivery tool shaft 1208. The majority of the delivery tool shaft, including the distal tip, is not shown. The resistive elements 1201 and 1202 are connected to the two conducting wires 1204 and 1205 respectively. The conducting wires run parallel down the inner lumen of the measuring cable 1206 and terminate at a point where they are connected to a measuring device such as a voltmeter, which is not shown.

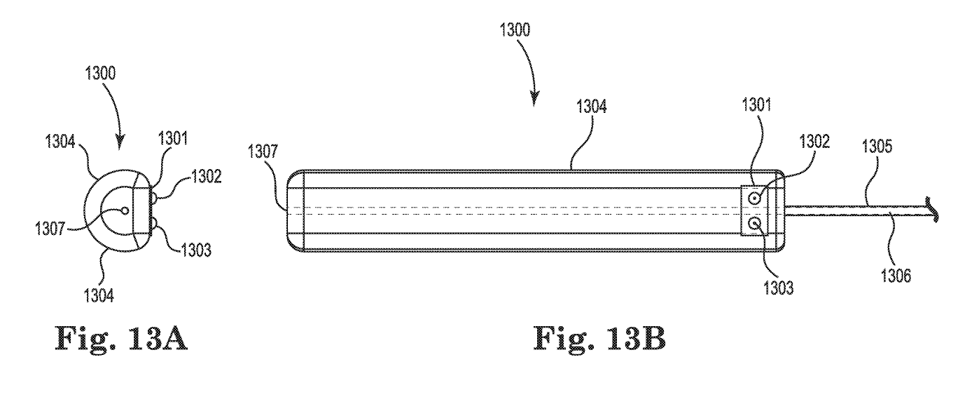

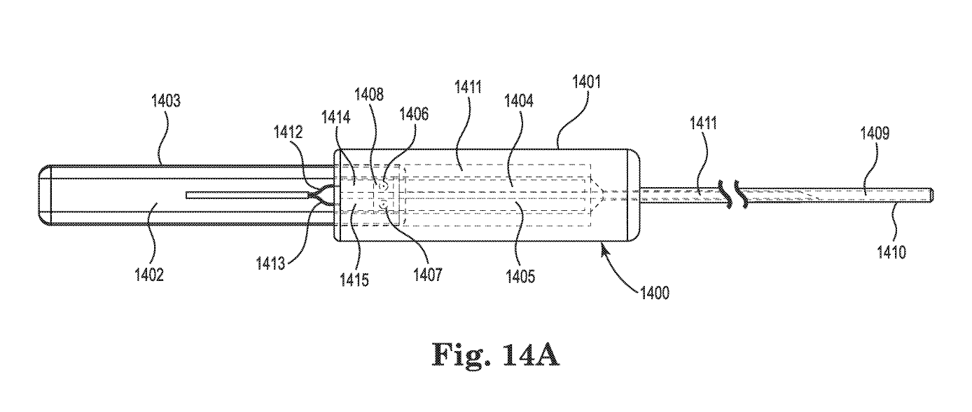

FIG. 13A and FIG. 13B are the proximal top and proximal side view respectively of the incorporation of the LPMM's wiper element 1301 into the transseptal needle 1300. The wiper element 1301 is a straight thin piece of conductive material such as but not limited to copper. On the wiper element 1301, there are two contact bumps 1302 and 1303 which function to make a sliding electrical connection to the two resistive elements 1201 and 1202 shown in FIG. 12A and FIG. 12B. The wiper element 1301 is located on the most distal aspect of the transseptal needle handle 1304. The wiper element 1301 could be adhesive bonded or mechanically attached to the needle handle 1304. Conversely, the needle handle 1304 could be overmolded onto the wiper element 1301. The transseptal needle cannula 1305 exits the distal side of the transseptal needle handle 1304. The transseptal needle inner lumen 1306 starts at the handle opening 1307 on the most proximal side of the needle handle 1304 and runs the length of the needle cannula 1305. The majority of the needle cannula, including the distal tip, is not shown.

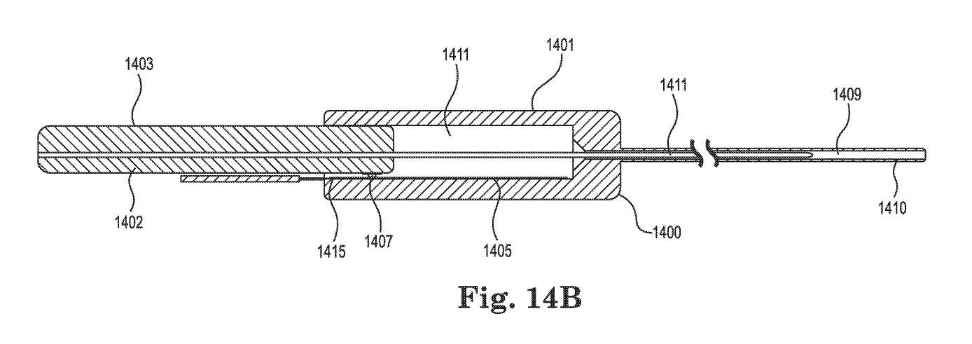

FIG. 14A and FIG. 14B are the top and side view respectively of the transseptal needle 1402 in a first position within the delivery tool 1400. In this position, the transseptal needle is almost fully advanced into the delivery tool and therefore the majority of the transseptal needle cannula 1411 is contained within the delivery tool shaft 1410 lumen 1409. Additionally, the distal edge of the transseptal needle handle 1403 has entered the delivery tool handle receiving cavity 1411. Finally, the contact bumps 1406, 1407 of the wiper element 1408 are in contact with the resistive elements 1404, 1405 respectively. With the contact bumps in contact with the resistive elements and the terminating ends of the conducting wires 1412, 1413 connected to a measuring device, which is not pictured, an electrical loop is formed. The amount of resistance within the electrical loop is a function of the length of the resistive elements 1414, 1415 contained within the electrical loop.

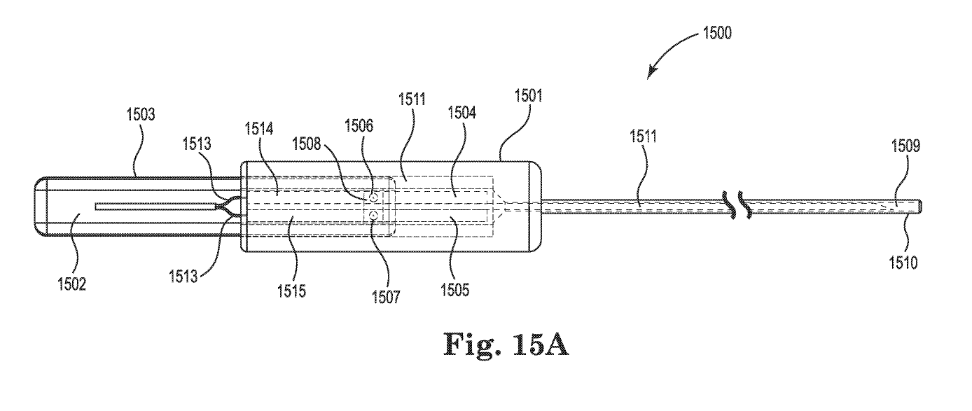

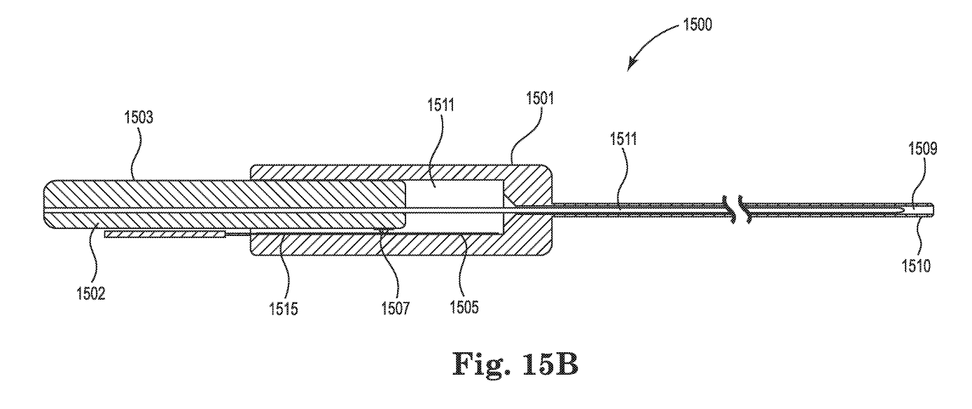

FIG. 15A and FIG. 15B are the top and side view respectively of the transseptal needle 1502 in a second position within the delivery tool 1500. In this position, the transseptal needle is more advanced into the delivery tool than in FIGS. 14A and 14B and the length of the resistive elements 1514, 1515 contained within the electrical loop is greater than the length of the resistive elements 1414, 1415 in FIG. 14A and FIG. 14B. Therefore, the resistance within the electrical loop in FIG. 15A and FIG. 15B is greater than the resistance in the electrical loop in FIG. 14A and FIG. 14B.

What is claimed:

1. An MR compatible RF transseptal needle system comprising:
a transseptal needle having a main cannula portion and a distal tip portion;
a delivery tool with a distal tip electrode.

2. The MR compatible RF transseptal needle system of claim 1 wherein the distal tip portion of the transseptal needle is a conductive material such as copper, gold, platinum iridium, stainless steel, MP35n, or the like.

3. The MR compatible RF transseptal needle system of claim 1 wherein the main cannula portion is comprised of an MR safe material such as PEEK, grilamid, polyimide, fiber, nylon, fiber re-enforced epoxy, ceramic, or some other polymer.

4. The MR compatible RF transseptal needle system of claim 1 wherein the delivery tool distal tip electrode is a conductive material such as copper, gold, platinum iridium, stainless steel, MP35, or the like.

5. The MR compatible RF transseptal needle system of claim 1 wherein the delivery tool distal tip electrode is a ring electrode.

6. The MR compatible RF transseptal needle system of claim 1 wherein the delivery tool distal tip electrode is positioned within the inner lumen of the delivery tool.

7. The MR compatible RF transseptal needle system of claim 1 wherein the delivery tool distal tip electrode is positioned at a tip of the delivery tool.

8. The MR compatible RF transseptal needle system of claim 1 wherein the delivery tool distal tip electrode is operably coupled to one or more conduction lines.

9. The MR compatible RF transseptal needle system of claim 1 wherein the delivery tool conduction lines are disposed within the wall of the delivery tool.

10. The MR compatible RF transseptal needle system of claim 1 wherein the delivery tool conduction lines are disposed within the inner lumen of the delivery tool.

11. The MR compatible RF transseptal needle system of claim 1 wherein the delivery tool conduction lines are positioned on the outside of the delivery tool.

12. The MR compatible RF transseptal needle system of claim 1 wherein the distal tip portion of the transseptal needle forms an electrical connection with the distal tip electrode of the delivery tool.

13. The MR compatible RF transseptal needle system of claim 1 wherein a fit between the distal tip portion of the transseptal needle and the distal tip electrode is selected from a compression fit, a sliding fit, or a stop fit.

14. The MR compatible RF transseptal needle system comprising:
a transseptal needle comprised of a main cannula portion and a distal tip portion.

15. The MR compatible RF transseptal needle system of claim 14 wherein the distal tip portion of the transseptal needle is comprised of a conductive material such as copper, gold, platinum iridium, stainless steel, MP35n, or the like.

16. The MR compatible RF transseptal needle system of claim 14 wherein the main cannula portion is comprised of an MR safe material such as PEEK, grilamid, polyimide, fiber, nylon, fiber re-enforced epoxy, ceramic, or some other polymer.

17. The MR compatible RF transseptal needle system of claim 14 wherein one or more conduction lines are electrically connected to the distal tip portion of the transseptal needle.

18. The MR compatible RF transseptal needle system of claim 1 wherein the delivery tool has one or more fiber optic bragg sensors disposed within the wall of the delivery tool.

19. The MR compatible RF transseptal needle system of claim 1 wherein the distal tip portion is slidably connected with the main cannula portion.

20. The MR compatible RF transseptal needle system of claim 1 wherein the distal tip portion is connected to a fiber optic bragg sensor

21. The MR compatible RF transseptal needle system of claim 1 further comprising a linear position measurement mechanism.

22. The MR compatible RF transseptal needle system of claim 21 wherein the linear position measurement mechanism comprises:
a wiper member; and
two or more conductive strip members.

23. The MR compatible RF transseptal needle system of claim 22 wherein the wiper member is comprised of a conductive material such as copper, gold, platinum iridium, stainless steel, MP35n, or the like.

24. The MR compatible RF transseptal needle system of claim 22 wherein the one or more conductive strip members are comprised of a conductive material such as copper, gold, platinum iridium, stainless steel, MP35n, or the like.

25. The MR compatible RF transseptal needle system of claim 22 wherein the wiper member forms a slidable electrical fit with the one or more conductive strip members.

26. The MR compatible RF transseptal needle system of claim 22 wherein an electrical loop is formed between the wiper member and conductive strip members.

27. The MR compatible RF transseptal needle system of claim 26 wherein the resistance within the electrical loop is measured.

28. The MR compatible RF transseptal needle system of claim 27 wherein the resistance is used to determine the linear relationship between the wiper member and the conductive strip members.

29. The MR compatible RF transseptal needle system of claim 22 wherein the wiper member is disposed on the transseptal needle handle and the conductive strip members are disposed on the delivery tool handle.

30. The MR compatible RF transseptal needle system of claim 22 wherein the wiper member is disposed on the delivery tool handle and the conductive strips are disposed on the transseptal needle handle.

31. The MR compatible RF transseptal needle system of claim 22 wherein the linear position measurement mechanism is positioned distally or proximally.
